SEQUENCE LISTING (28 sequences)

SEQ ID NO: 1 - protein, 278 aa, Artificial Sequence
  Bacillus subtilis(Bs) flagellin
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRPGK

SEQ ID NO: 2 - protein, 505 aa, Artificial Sequence
  Salmonella dublin(Sd) flagellin
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAVAPDKVYVNAANGQLTTDDAENNTAVDLFKTTKSTAGTAEAKAIAGAIKGGKEGDTFDYKGVTFTIDTKTGDDGNGKVSTTINGEKVTLTVADIAIGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 3 - protein, 387 aa, Artificial Sequence
  Pseudomonas aeruginosa (Pa) flagellin
MALTVNTNIASLNTQRNQNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIDITGGSAVNVKVDMKGNETAEQAAAKIAAAVNDANVGIGAFTDGAQISYVSKASADGTTSAVSGVAITDTGSTGAGTAAGTTTFTEANDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 4 - protein, 550 aa, Artificial Sequence
  Shigella flexneri (Sf) flagellin
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGGGAVANTAASKADLVAANATVVGNKYTVSAGYDAAKASDLLAGVSDGDTVQATINNGFGTAASATNYKYDSASKSYSFDTTTASAADVQKYLTPGVGDTAKGTITIDGSAQDVQISSDGKITASNGDKLYIDTTGRLTKNGSGASLTEASLSTLAANNTKATTIDIGGTSISFTGNSTTPDTITYSVTGAKVDQAAFDKAVSTSGNNVDFTTAGYSVNGTTGAVTKGVDSVYVDNNEALTTSDTVDFYLQDDGSVTNGSGKAVYKDADGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQG

SEQ ID NO: 5 - protein, 498 aa, Artificial Sequence
  Escherichia coli (Ec) flagellin
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 6 - protein, 229 aa, Artificial Sequence
  Human IgG4 Fc region
AMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 227 aa, Artificial Sequence
  Mouse IgG1 Fc region
TMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 5 aa, Artificial Sequence
  Linker 1
GGGGS

SEQ ID NO: 9 - protein, 15 aa, Artificial Sequence
  Linker 2
GGGGSGGGGSGGGGS

SEQ ID NO: 10 - protein, 509 aa, Artificial Sequence
  hIgG4-Fc-Bsflagellin fusion protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 507 aa, Artificial Sequence
  mIgG1-Fc-Bsflagellin fusion protein (with linker 1)
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSTMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 517 aa, Artificial Sequence
  mIgG1-Fc-Bsflagellin fusion protein (with linker 2)
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSGGGGSGGGGSTMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 761 aa, Artificial Sequence
  hIgG4-Fc-Sdflagellin  fusion protein (with linker 1)
MYRMQLLSCIALSLALVTNSISMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAVAPDKVYVNAANGQLTTDDAENNTAVDLFKTTKSTAGTAEAKAIAGAIKGGKEGDTFDYKGVTFTIDTKTGDDGNGKVSTTINGEKVTLTVADIAIGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 643 aa, Artificial Sequence
  hIgG4-Fc-Paflagellin  fusion protein (with linker 1)
MYRMQLLSCIALSLALVTNSISMALTVNTNIASLNTQRNQNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIDITGGSAVNVKVDMKGNETAEQAAAKIAAAVNDANVGIGAFTDGAQISYVSKASADGTTSAVSGVAITDTGSTGAGTAAGTTTFTEANDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 806 aa, Artificial Sequence
  hIgG4-Fc-Sfflagellin fusion protein (with linker 1)
MYRMQLLSCIALSLALVTNSISMAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGGGAVANTAASKADLVAANATVVGNKYTVSAGYDAAKASDLLAGVSDGDTVQATINNGFGTAASATNYKYDSASKSYSFDTTTASAADVQKYLTPGVGDTAKGTITIDGSAQDVQISSDGKITASNGDKLYIDTTGRLTKNGSGASLTEASLSTLAANNTKATTIDIGGTSISFTGNSTTPDTITYSVTGAKVDQAAFDKAVSTSGNNVDFTTAGYSVNGTTGAVTKGVDSVYVDNNEALTTSDTVDFYLQDDGSVTNGSGKAVYKDADGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQGGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 754 aa, Artificial Sequence
  hIgG4-Fc-Ecflagellin fusion protein (with linker 1)
MYRMQLLSCIALSLALVTNSISMAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQGGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 2 aa, Artificial Sequence
  peptide linker 1
GG

SEQ ID NO: 18 - protein, 3 aa, Artificial Sequence
  peptide linker 2
GAG

SEQ ID NO: 19 - protein, 3 aa, Artificial Sequence
  peptide linker 3
GPA

SEQ ID NO: 20 - protein, 1 aa, Artificial Sequence
  peptide linker 4
G

SEQ ID NO: 21 - protein, 2 aa, Artificial Sequence
  peptide linker 5
GS

SEQ ID NO: 22 - protein, 5 aa, Artificial Sequence
  peptide linker 6
GSGSG

SEQ ID NO: 23 - protein, 3 aa, Artificial Sequence
  peptide linker 7
GGS

SEQ ID NO: 24 - protein, 5 aa, Artificial Sequence
  peptide linker 8
GGSSG

SEQ ID NO: 25 - protein, 4 aa, Artificial Sequence
  peptide linker 9
GGSG

SEQ ID NO: 26 - protein, 4 aa, Artificial Sequence
  peptide linker 10
GGGS

SEQ ID NO: 27 - protein, 6 aa, Artificial Sequence
  peptide linker 11
GGSGGG

SEQ ID NO: 28 - protein, 13 aa, Artificial Sequence
  peptide linker 12
GSGGGGSGGGGSG

(19) United States
(12) Patent Application Publication
(10) Pub. No.: US 2024/0270798 A1
(43) Pub. Date: Aug. 15, 2024

(54) COMPOSITION FOR PREVENTING OR TREATING NON-ALCOHOLIC FATTY LIVER DISEASE OR METABOLIC SYNDROME, COMPRISING FLAGELLIN FUSION PROTEINS

(71) Applicants: MEDISPAN CO., LTD, Gyeonggi-do (KR); INDUSTRY FOUNDATION OF CHONNAM NATIONAL UNIVERSITY, Gwangju (KR)

(72) Inventors: Kyung A. CHO, Gwangju (KR); Jae Sung LIM, Gwangju (KR)

(21) Appl. No.: 18/032,902

(22) PCT Filed: Jul. 16, 2021
(86) PCT No.: PCT/KR2021/009151

§ 371 Date: Apr. 20, 2023

(30) Foreign Application Priority Data
Oct. 20, 2020 (KR) 10-2020-0136280

(51) Int. Cl. C07K 14/195; A61K 38/00; A61P 1/16; A61P 3/04; A61P 3/10

(52) U.S. Cl. CPC C07K 14/195; A61P 1/16; A61P 3/04; A61P 3/10; A61K 38/00; C07K 2319/30

(57) ABSTRACT

The present invention relates to a composition for preventing or treating a non-alcoholic fatty liver disease or metabolic syndrome, comprising flagellin fusion proteins and, more specifically, to a composition for preventing or treating a non-alcoholic fatty liver disease or metabolic syndrome, comprising fusion proteins comprising: flagellin, a fragment thereof, or a variant thereof; and an immunoglobulin Fc region.

This application is a National Phase Application of PCT/KR2021/009151, filed Jul. 16, 2021, which claims priority to Korean Patent Application No. 10-2020-0136280, filed on Oct. 20, 2020, and the entire specifications are incorporated herein by reference.

### REFERENCE TO A SEQUENCE LISTING

A Sequence Listing conforming to the rules of WIPO Standard ST.25 is hereby incorporated by reference. Said Sequence Listing has been filed as an electronic document via PatentCenter in ASCII formatted text. The electronic document, created on Oct. 19, 2023, is entitled “11239-016US1_ST25”, and is 60,292 bytes in size.

### TECHNICAL FIELD

The present invention relates to a composition for the prevention or treatment of non-alcoholic fatty liver disease or metabolic syndrome, which includes a fusion protein of flagellin. More specifically, it relates to a composition for preventing or treating non-alcoholic fatty liver disease or metabolic syndrome, including flagellin, fragments or variants thereof; and fusion proteins including the immunoglobulin Fc region.

### TECHNOLOGIES UNDERLYING THE INVENTION

Metabolic syndrome is a group of disorders that occur due to metabolic dysfunction within the body. It typically arises from imbalances in substances such as carbohydrates, lipids, proteins, vitamins, electrolytes, and water, and can lead to conditions such as obesity, diabetes, hyperlipidemia, fatty liver, atherosclerosis, and hypertension. In the case of type 2 diabetes, which mainly occurs in adulthood, there is an increase in resistance to insulin. This can occur when there is a decrease in insulin receptors, a decrease in their sensitivity, or when there are problems with the secondary messengers that cause glycogen synthesis in cells. Therefore, type 2 diabetes is called Non-insulin dependent diabetes and accounts for 85˜90% of diabetes.

Obesity is particularly concerning not only due to its social and aesthetic implications but also because it can lead to serious health risks such as metabolic complications including diabetes and hypertension. One such symptom related to the pathological state of obesity is systemic chronic inflammation, which occurs in obese individuals. Inflammation is one of the immune mechanisms that occurs in the body and is an important response to defend against pathogens or viruses that invade the body when it occurs locally. However, when this inflammation response is excessively and chronically activated due to the collapse of the balance of immune responses in the body, it can interfere with metabolic processes in the body. Chronic inflammation caused by obesity has been identified as the cause of various metabolic disorders such as diabetes, cardiovascular disease, and atherosclerosis. It is also considered the most important factor in defining obesity as a disease. Obesity is simply a cosmetic problem without the onset of secondary metabolic syndrome caused by chronic inflammatory reactions, and the recently, World Health Organization has recently defined obesity as a disease, citing chronic inflammatory reactions that can significantly reduce the quality of life, such as diabetes.

Meanwhile, fatty liver refers to a pathological state where abnormal fat accumulates within liver cells, medically indicating a condition where the neutral lipid content exceeds 5% of the total liver weight. Generally, fatty liver can be classified into two types: alcoholic fatty liver disease (ALD) caused by continuous and excessive alcohol consumption, and non-alcoholic fatty liver disease (NAFLD) which is characterized by similar liver tissue findings as ALD, but with little to no alcohol intake.

ALD is common in South Korea and occurs due to alcohol-induced hepatic lipid synthesis and disrupted normal energy metabolism, which can progress to hepatitis or liver cirrhosis depending on the amount of alcohol consumed.

NAFLD can occur due to various causes such as obesity, diabetes, hyperlipidemia, and medication, regardless of alcohol consumption. It encompasses a wide range of diseases, including simple steatosis without inflammation, non-alcoholic steatohepatitis (NASH) with hepatocellular inflammation, advanced fibrosis, and cirrhosis, depending on the disease progression.

Non-alcoholic fatty liver disease (NAFLD) is an increase in adult diseases due to high-fat and high-calorie diet intake in modern society, and 20˜30% of the adult population in developed countries has non-alcoholic fatty liver disease (NAFLD), of which 2-3% are non-alcoholic steatohepatitis (NASH) patients have been reported. In particular, the histological findings of steatohepatitis accompanied by fibrosis and inflammation show a very high risk of developing liver cirrhosis, liver failure, and liver cancer.

Although the relationship between fatty liver disease and obesity, insulin resistance, and type 2 diabetes has been established, the exact mechanism of onset is still under active investigation. The ‘two-hit’ hypothesis suggests that steatohepatitis begins when fat accumulates in hepatocytes, and insulin resistance associated with obesity and diabetes is considered to be the main pathogenesis. In addition, it has been reported that lipotoxicity caused by increased free fatty acid (FFA) or cholesterol in hepatocytes, increased inflammatory cytokines, and these receptors play an important role in the process of progressing from fatty liver to steatohepatitis.

The steady increase in high-energy food consumption and social changes associated with reduced physical activity are contributing to the rising prevalence of obesity and metabolic syndrome. Traditional therapies based on low-calorie diets and exercise have not been very effective in controlling obesity and have only resulted in temporary weight loss. The development of safe and effective drugs for inducing weight loss has been ongoing for many years, but so far, drugs that have demonstrated efficacy have also caused serious side effects or had limited efficacy in clinical trials. Therefore, new approaches are needed to improve obesity and metabolic syndrome and prevent these pathological conditions.

### DESCRIPTION OF INVENTION

### Problem to be Solved

As a result of repeated research to develop a substance that can effectively prevent or treat non-alcoholic fatty liver disease or metabolic syndrome, the present inventor discovered that a novel fusion protein consisting of flagellin and immunoglobulin Fc showed excellent therapeutic effects for non-alcoholic fatty liver disease and metabolic syndrome. Thus, the present invention was completed.

Accordingly, an object of the present invention is to provide a pharmaceutical composition for the prevention or treatment of non-alcoholic fatty liver disease or metabolic syndrome comprising as an active ingredient a fusion protein comprising flagellin, a fragment or a variant thereof and an immunoglobulin Fc region.

In addition, an object of the present invention is to provide a pharmaceutical composition for the prevention or treatment of non-alcoholic fatty liver disease or metabolic syndrome consisting of as an active ingredient a fusion protein comprising flagellin, a fragment or a variant thereof and an immunoglobulin Fc region.

In addition, an object of the present invention is to provide a pharmaceutical composition for the prevention or treatment of non-alcoholic fatty liver disease or metabolic syndrome, essentially consisting of as an active ingredient a fusion protein comprising flagellin, a fragment or a variant thereof and an immunoglobulin Fc region.

Another object of the present invention is to provide a food composition for the prevention or improvement of non-alcoholic fatty liver disease or metabolic syndrome comprising a fusion protein comprising flagellin, a fragment or a variant thereof, and an immunoglobulin Fc region as an active ingredient.

In addition, another object of the present invention is to provide a food composition for the prevention or improvement of non-alcoholic fatty liver disease or metabolic syndrome consisting of a fusion protein comprising flagellin, a fragment or a variant thereof, and an immunoglobulin Fc region as an active ingredient.

In addition, another object of the present invention is to provide a food composition for the prevention or improvement of non-alcoholic fatty liver disease or metabolic syndrome, essentially consisting of a fusion protein comprising flagellin, a fragment or a variant thereof, and an immunoglobulin Fc region as an active ingredient.

Another object of the present invention is to provide a use of a fusion proteins comprising flagellin, a fragment, or variant thereof, and an immune globulin Fc domain, for the preparation of an agent for the treatment of non-alcoholic fatty liver disease or metabolic syndrome.

Another object of the present invention is to provide a method for treating non-alcoholic fatty liver disease or metabolic syndrome, the method comprising administering an effective amount of a composition comprising a fusion protein comprising flagellin, a fragment or a variant thereof, and an immunoglobulin Fc region as an active ingredient to a subject in need thereof.

### Means for Solving the Problem

In order to achieve the above-identified object of the present invention provides a pharmaceutical composition for the prevention or treatment of non-alcoholic fatty liver disease or metabolic syndrome comprising a fusion protein comprising flagellin, a fragment or a variant thereof and an immunoglobulin Fc region.

In addition, the present invention provides a pharmaceutical composition for the prevention or treatment of non-alcoholic fatty liver disease or metabolic syndrome consisting of as an active ingredient a fusion protein comprising flagellin, a fragment or a variant thereof and an immunoglobulin Fc region.

In addition, the present invention provides a pharmaceutical composition for the prevention or treatment of non-alcoholic fatty liver disease or metabolic syndrome, essentially consisting of as an active ingredient a fusion protein comprising flagellin, a fragment or a variant thereof and an immunoglobulin Fc region.

To achieve another object of the present invention, the present invention provides a food composition for the prevention or improvement of non-alcoholic fatty liver disease or metabolic syndrome comprising a fusion protein comprising flagellin, a fragment or a variant thereof, and an immunoglobulin Fc region as an active ingredient.

In addition, the present invention provides a food composition for the prevention or improvement of non-alcoholic fatty liver disease or metabolic syndrome consisting of a fusion protein comprising flagellin, a fragment or a variant thereof, and an immunoglobulin Fc region as an active ingredient.

In addition, the present invention provides a food composition for the prevention or improvement of non-alcoholic fatty liver disease or metabolic syndrome, essentially consisting of a fusion protein comprising flagellin, a fragment or a variant thereof, and an immunoglobulin Fc region as an active ingredient.

To achieve another object of the present invention, the present invention provides a use of a fusion proteins comprising flagellin, a fragment, or variant thereof, and an immune globulin Fc domain, for the preparation of an agent for the treatment of non-alcoholic fatty liver disease or metabolic syndrome.

To achieve another object of the present invention, the present invention provides a method for treating non-alcoholic fatty liver disease or metabolic syndrome, the method comprising administering an effective amount of a composition comprising a fusion protein comprising flagellin, a fragment or a variant thereof, and an immunoglobulin Fc region as an active ingredient to a subject in need thereof.

Hereinafter, the present invention will be described in detail.

The present invention provides a pharmaceutical composition for the prevention or treatment of non-alcoholic fatty liver disease or metabolic syndrome comprising as an active ingredient a fusion protein comprising flagellin, a fragment or a variant thereof and an immunoglobulin Fc region.

In the present invention, the flagellin can induce an immune response within an infected host when a flagellated bacterium is present. More specifically, toll-like receptor 5 (TLR5), present on the surface of the cell membrane, can interact with the flagellin to trigger intracellular signaling, which leads to increased expression of the transcription factor NF-kB, thereby activating innate immune signaling and regulating acquired immune responses.

The flagellin protein is well known, for example, from U.S. Pat. Nos. 6,585,980, 6,130,082, 5,888,810, 5,618,533, 4,886,748, and US Patent Application Publication No. US2003/0044429 A1, as well as from Donnelly et al. (2002) J. Biol. Chem. 43:4045. Most Gram-negative bacteria express flagella, which are surface structures that provide motility. The flagellum is composed of a basal body, filament, and hook that connects them. The filaments are made of long polymers of a single protein, flagellin, with a small cap protein at the end.

While the Polymerization of flagellin is mediated by conserved regions at the N- and C-termini, the hypervariable region located in the middle of the flagellin protein varies greatly in sequence and length between different species.

In the present invention, the flagellin may be flagellin derived from any suitable bacteria. Many flagellin genes have been cloned and sequenced in the art and may be referenced.

As an unrestricted supply source of the flagellin in this invention, microorganisms belonging to the Bacillus, Salmonella, Helicobacter, Vibrio, Serratia, Shigella, Treponema, Legionella, Borrelia, Clostridium, Agrobacterium, Bartonella, Proteus, Pseudomonas, Escherichia, Listeria, Yersinia, Campylobacter, Roseburia, or Marinobacter genus can be used, with Bacillus, Salmonella, or Vibrio microorganisms being preferred.

More preferably, the flagellin in the present invention may be derived from Salmonella enteritidis, Salmonella typhimurium, Salmonella dublin, Salmonella enterica, Helicobacter pylori, Vibrio cholera, Vibrio vulnificus, Vibrio fibrisolvens, Serratia marcesens, Shigella flexneri, Treponema pallidum, Borrelia burgdorferei, Clostridium difficile, Agrobacterium tumefaciens, Bartonella clarridgeiae, Proteus mirabilis, Bacillus subtilis, Bacillus cereus, Bacillus halodurans, Pseudomonas aeruginosa, Escherichia coli, Listeria monocytogenes, Yersinia pestis, Campylobacter spp, Roseburia spp, or Marinobacter spp, and

More preferably, the flagellin in the present invention may be derived from Salmonella enteritidis, Salmonella typhimurium, Salmonella dublin, Salmonella enterica, Vibrio cholera, Vibrio vulnificus, Vibrio fibrisolvens, Bacillus subtilis, Bacillus cereus, or Bacillus halodurans, and

Most preferably, the flagellin in the present invention may be derived from Bacillus subtilis.

TLR5 recognizes evolutionarily conserved regions of bacterial flagellin required for flagellar filament assembly and movement, but flagellin of some α and ε Proteobacteria (Campylobacter jejuni, Helicobacter pylori, and Bartonella bacilliformis) is not recognized by TLR5, as reported in the following papers [(2003) Microbes Infect. 5, 1345-1356; J. Infect. Dis. 189, 1914-1920; (2002) Nat. Rev. Cancer 2, 28-37; (2001) Clin. Infect. Dis. 32, 1201-1206. pmid:11283810]. Bacterial flagellins recognized by TLR5 have similarly conserved major amino acid sequences in the D0 and D1 domains, while bacterial flagellins not recognized by TLR5 have different amino acid sequences (PNAS Jun. 28, 2005 102 (26) 9247-9252; Scientific Reports|7:40878|DOI: 10.1038/srep40878). Therefore, in the present invention, the flagellin may contain conserved sequences recognized by TLR5 in the D0 and D1 domains.

The present inventor has previously produced Fc-fusion proteins of the flagellins of five representative bacteria, whose flagellins contain conserved amino acid sequences recognized by TLR5 in the D0 and D1 domains, based on previously reported papers (Korean Patent Application No. 10-2020-0048368). In an embodiment of the present invention, the therapeutic effect of the flagellin-Fc fusion protein was confirmed in the treatment of metabolic syndrome.

The N-terminal and C-terminal constant regions of flagellin are well characterized in the art and are described, for example, in Mimori-Kiyosue et al. (1997) J. Mol. Biol. 270: 222-237; Iino et al. (1977) Ann. Genet. 11: 161-182; and Schoenhals et al. (1993) J. Bacteriol. 175: 5395-5402. As is well understood by those skilled in the art, the size of the constant region can vary somewhat depending on the source of the flagellin protein. Generally, N-terminal constant domains include approximately 170 or 180 N-terminal amino acids of the protein, whereas the C-terminal constant domains typically include about 85 to 100 C-terminal amino acids. The central hypervariable region varies considerably depending on the size and order of bacteria, and most of the differences in molecular weight can be attributed to this hypervariable region. The N- and C-terminal constant regions of flagellin proteins derived from various bacteria are known, and flagellin derived from bacteria that are not yet known can easily identify the crystal structure of flagellin monomers using techniques known in the art.

In the present invention, the terms “flagellin,” “flagellin N-terminal constant region,” and “flagellin C-terminal constant region” include flagellin active fragments and variants derived from any of the bacteria exemplified herein. Furthermore, the wild-type flagellin or a portion thereof may be modified for increased safety and/or immunogenicity or as a result of cloning procedures or other laboratory manipulations, and such modifications (or variants) are also within the scope of the present invention.

In the present invention, the flagellin may include full-length flagellin or an active fragment. In addition, terms such as “flagellin”, “flagellin N-terminal constant region” and “flagellin C-terminal constant region” may include naturally occurring amino acid sequences. It may also include amino acid sequences that are substantially the same as or similar to amino acid sequences in naturally occurring flagellin, flagellin N-terminal constant regions, or flagellin C-terminal constant regions, respectively.

In the present invention, flagellin, flagellin N-terminal constant region, flagellin C-terminal constant region, or the “active fragment” of any other portion of flagellin may include at least about 50, 75, 100, 125, 150, 200, 250, or 300 adjacent amino acids and/or fewer than about 300, 250, 200, 150, 125, 100, or 75 adjacent amino acids. If the lower limit is smaller than the upper limit, a combination thereof may also be included. Such active fragment may represent a fragment capable of activating the TLR5 pathway in the host.

In certain embodiments, the active fragment can activate the TLR5 pathway as at least about 50%, 75%, 80%, 85%, 90%, or 95% of the full-length flagellin. The TLR5 pathway can be activated to the same or essentially the same extent as the full-length flagellin or flagellin site, or the TLR5 pathway can be activated to a higher degree compared to the full-length flagellin or flagellin site.

In the present invention, the active fragment may mean at least a portion of flagellin that exhibits activity in the TLR5 pathway. The term “at least a portion” may refer to a region of flagellin that exhibits activity in the TLR5 pathway in domains 0, 1, 2, and 3. Specifically, the active fragment may be flagellin that the hypervariable region has been removed. The hypervariable region may vary depending on the type of bacteria from which flagellin is derived, and among the entire sequence of specific flagellin, a sequence corresponding to the hypervariable region can be easily identified and removed by a person skilled in the art. For example, in the case of full-length flagellin including N-terminal domains 0, 1, 2, domain 3, and C-terminal domains 2, 1, 0, domain 3 or domains 2 and 3 may be the hypervariable region, and in the case of full-length flagellin including N-terminal domains 0, 1, domain 2, C-terminal domains 1, 0, domain 2 may be the hypervariable region. Alternatively, in the case of flagellin that does not contain a hypervariable region (e.g., flagellin derived from many Gram-positive bacteria may not contain a hypervariable region), a portion of the hinge region of the flagellin protein where folding occurs may have been partially removed.

The term “hypervariable region” used in the present invention may be expressed as a propeller domain or region, a hinge, a hypervariable region, a variable domain or region, and the like.

In present invention, the deletion of the hypervariable region may mean that the entire domain corresponding to the hypervariable region or the partial removal of some of the sequences within the hypervariable region.

In present invention, the active fragment may be flagellin in which the hypervariable region of wild-type flagellin is removed and an artificial sequence (i.e., a hinge or linker of the artificial sequence) is inserted into the removed hypervariable region.

The flagellin fragment of the present invention may mean a fragment showing TLR5 pathway activity while including at least one selected from the group consisting of a C-terminal domain 0, a C-terminal domain 1, a C-terminal domain 2, an N-terminal domain 2, an N-terminal domain 1, an N-terminal domain 0, and a region showing 80% or more amino acid sequence homology with each of the domains.

In certain embodiments, the active fragment can activate the TLR5 pathway as at least about 50%, 75%, 80%, 85%, 90%, or 95% of the full-length flagellin. The TLR5 pathway can be activated to the same or essentially the same extent as the full-length flagellin or flagellin site, or the TLR5 pathway can be activated to a higher degree compared to the full-length flagellin or flagellin site.

The present invention also includes proteins having the full-length sequence of wild-type flagellin as well as amino acid sequence variants thereof. In the present invention, a variant means a protein having a different sequence by deletion, insertion, non-conservative or conservative substitution, substitution of an amino acid analog or a combination of thereof. Amino acid substitutions that do not entirely alter the activity of the molecule (i.e., its ability to activate the TLR5 pathway) are known in the art (H. Neurath, R. L. Hill, The Proteins, Academic Press, New York, 1979).

In some cases, the variant of the present invention may be a full-length flagellin or fragment thereof modified by phosphorylation, sulfation, acrylation, glycosylation, methylation, farnesylation, or the like.

In certain embodiments, the variant of flagellin or a fragment thereof can activate the TLR5 pathway as at least about 50%, 75%, 80%, 85%, 90%, or 95% of the full-length flagellin. The TLR5 pathway can be activated to the same or essentially the same extent as the full-length flagellin or flagellin site, or the TLR5 pathway can be activated to a higher degree compared to the full-length flagellin or flagellin site.

In the present invention, the flagellin, its fragment, or its variant may be in the form of a fusion protein containing other polypeptides. For example, the flagellin may be a fusion protein containing one or more antigens. Non-limiting examples of such antigens may be included S. pneumoniae PspA1 antigen, S. pneumoniae PspA2 antigen, S. pneumoniae PspA3 antigen, S. pneumoniae PspA4 antigen, S. pneumoniae PspA5 antigen, and/or S. pneumoniae PspA6 antigen. Alternatively, for example, the flagellin may be in the form of a fusion protein in which one or more immunomodulatory substances are combined. The immunomodulatory substance may be included without limitation as long as it is known to increase the immune response in the art, and non-limiting examples thereof are interferon-α, interferon-β, interferon-γ, interferon-ω, interferon-τ, interleukin-1α, interleukin-1β, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-18, B-cell growth factor, CD40 ligand, TNF-α, TNF-β, CCL25, CCL28, or an active fragment thereof.

The term “percent (%) sequence identity” used in this invention is defined as the percentage of amino acid residues in a candidate sequence that are identical to amino acid residues in a reference polypeptide. After aligning the sequences and introducing gaps, any conservative substitutions are not considered part of the sequence identity, if necessary, to achieve the maximum percent sequence identity. Alignment for the purpose of determining percent amino acid homology can be performed using a variety of methods and methods within the skill of the art using, for example, publicly available computer software programs such as BLAST, BLAST-2, ALIGN) or Megalign (DNASTAR) software. One skilled in the art can determine appropriate parameters for measuring alignment, including any algorithms necessary to achieve maximal alignment over the entire length of the sequences being compared. For purposes herein, the percent (%) amino acid sequence identity of a given amino acid sequence A to a given amino acid sequence B or to a given amino acid sequence B is calculated as follows: 100 times fraction X/Y, where X is the number of amino acid residue scores identically matched by the sequence alignment program in program alignments of A and B, and Y is the total number of amino acid residues in B. It will be appreciated that if the length of amino acid sequence A is not equal to the length of amino acid sequence B, then the percent (%) amino acid sequence identity of A to B is not equal to the percent (%) amino acid sequence identity of B to A.

In a particular embodiment, the flagellin or its fragment or variant may be composed of an amino acid sequence selected from the group consisting of SEQ ID NO: 1 to 5, or an amino acid sequence showing at least 80% sequence identity with them.

In the present invention, the “Fc region” means the C-terminal part of the immunoglobulin heavy chain constant region, and specifically means the immunoglobulin heavy chain constant region or a part thereof.

The immunoglobulin Fc region of the present invention includes a natural amino acid sequence as well as a sequence mutant thereof. An amino acid sequence derivative means that one or more amino acid residues in a natural amino acid sequence have a different sequence by deletion, insertion, non-conservative or conservative substitution, or a combination thereof. For example, in the case of IgG Fc, amino acid residues 214 to 238, 297 to 299, 318 to 322 or 327 to 331, which are known to be important for binding, can be used as suitable sites for modification. Furthermore, various kinds of derivatives are possible, such as removing a site capable of forming a disulfide bond, removing some amino acids at the N-terminus from the natural type Fc, or adding a methionine residue to the N-terminus of the natural type Fc. In addition, in order to eliminate the effector function, a complement binding site, eg., a c1q binding site, may be removed, or an ADCC site may be removed. Techniques for preparing such immunoglobulin Fc region sequence derivatives are disclosed in International Patent Publication No. 97/34631, International Patent Publication No. 96/32478, and the like.

Amino acid substitutions in proteins and peptides that do not globally alter the activity of the molecule are well known in the art. The most commonly occurring substitutions are those between amino acid residues Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

In some cases, it can be modified by phosphorylation, sulfation, acrylation, glycosylation, methylation, farnesylation, acetylation, amidation, and the like.

The Fc derivatives described above show the same biological activity as the Fc region of the present invention, while enhancing the structural stability of the Fc region with respect to factors such as temperature and pH.

Additionally, these Fc regions can be obtained from natural forms isolated from animals such as humans, cows, goats, pigs, mouse, rabbits, hamsters, rats, and guinea pigs, or from recombinant forms or derivatives thereof derived from transformed animal cells or microorganisms. Here, the method of obtaining from the natural form can be obtained by isolating whole immunoglobulin from a human or animal body and then treating it with a proteolytic enzyme. When treated with papain, it is cleaved into Fab and Fc, and when treated with pepsin, it is cleaved into pF′c and F(ab)2. Fc or pF′c can be separated from this using size-exclusion chromatography and other techniques.

In addition, the Fc region of immunoglobulin can be in the form of natural glycosylation, increased or decreased glycosylation compared to natural forms, or in a form with removed glycosylation. Common methods such as chemical, enzymatic, and genetic engineering using microorganisms can be used to increase, decrease, or remove glycosylation in these Fc regions. Here, immunoglobulin Fc regions with removed glycosylation have significantly reduced complement (c1q) binding and decreased or removed antibody-dependent or complement-dependent cellular cytotoxicity, thereby not inducing unnecessary immune responses in the body. In this regard, Fc regions of immunoglobulin with removed or deglycosylated forms are more suitable for their original purpose as drug carriers.

In the present invention, “deglycosylation” refers to an IgG4 Fc variant where the sugar has been removed by an enzyme, while “aglycosylation” refers to a variant that is not glycosylated, produced in prokaryotes, preferably E. coli.

In addition, the immunoglobulin Fc region may be an Fc region derived from IgG, IgA, IgD, IgE, or IgM, a combination thereof, or a hybrid thereof. It is preferably derived from IgG or IgM, which is most abundant in human blood. Most preferably it may be from IgG, which is known to enhance the half-life of ligand binding proteins.

Meanwhile, in the present invention, combination refers to the formation of oligomers or polymers by binding poly-peptides that encrypt identical origin Fc regions with poly-peptides of different origin chains. That is, the manufacture of dimers or multimers from two or more fragments selected from a group consisting of Fc fragments of IgG, IgA, IgM, IgD, and IgE.

In the invention, “hybrid” refers to a term that signifies the presence of two or more different origin immune globulin Fc fragments within the Fc region of a monomer. Various forms of hybrids are possible in this invention. That is, hybrids consisting of one to four domains, including the hinge, are possible from the CH1, CH2, CH3, and CH4 of a group consisting of IgG Fc, IgM Fc, IgA Fc, IgE Fc, and IgD Fc.

On the other hand, IgG can also be divided into subclasses IgG1, IgG2, IgG3, and IgG4, and in the present invention, combinations of these or their hybridization are possible. Ideally, it may be derived from the Fc of human immunoglobulin IgG1.

In another embodiment, the Fc region of the immunoglobulin may include one or more selected from the group consisting of the CH1, CH2, CH3, and CH4 domains of the heavy chain and constant region of the immunoglobulin. For example, the Fc region of the immunoglobulin that can be used for the production of the fusion protein of the present invention may include (a) the CH1, CH2, CH3, and CH4 domains; (b) the CH1, CH2, and CH3 domains; (c) the CH1, CH2, and CH4 domains; (d) the CH1, CH3, and CH4 domains; (e) the CH2, CH3, and CH4 domains; (f) the CH1 and CH4 domains; (g) the CH1 and CH3 domains; (h) the CH1 and CH2 domains; (i) the CH2 and CH4 domains; (j) the CH2 and CH3 domains; (k) the CH3 and CH4 domains; or a combination of two or more domains and the hinge region of the immunoglobulin. Most preferably, the Fc region of the immunoglobulin may include the CH2 and CH3 domains of the heavy chain and constant region.

This invention includes other Fc regions of immunoglobulins, or their fragment sequences, which are encoded by nucleotide sequences disclosed in GenBank and/or EMBL databases, in addition to the human immunoglobulin Fc region. For example, such sequences may include AF045536.1 (Macaca fuscicularis), AF045537.1 (Macaca mulatta), AB016710 (Felix catus), K00752 (Oryctolagus cuniculus), U03780 (Sus scrofa), Z48947 (Camelus dromedarius), X62916 (Bos taurus), L07789 (Mustela vision), X69797 (Ovis aries), U17166 (Cricetulus migratorius), X07189 (Rattus rattus), AF57619.1 (Trichosurus vulpecula), or AF035195 (Monodelphis domestica), among others.

In the present invention, the above fusion proteins may be those in which the N-terminus or C-terminus of the flagellin, its fragment, or its variant is bound to the N-terminus or C-terminus of the immunoglobulin Fc region. Specifically, the N-terminus of the flagellin, its fragment, or its variant may be bound to the C-terminus of the immunoglobulin Fc region, or the C-terminus of the flagellin, its fragment, or its variant may be bound to the N-terminus of the immunoglobulin Fc region. Preferably, the C-terminus of the flagellin, its fragment, or its variant may be bound to the N-terminus of the immunoglobulin Fc region.

In certain embodiments, the immunoglobulin Fc region of the present invention may comprise an amino acid sequence of SEQ ID No: 6 or 7, or an amino acid sequence showing at least 80% sequence homology thereto.

Meanwhile, each component constituting the fusion protein in the present invention, namely flagellin, its fragment or variant, and the immunoglobulin Fc region, can be directly connected or connected through a linker. Generally, the term “linker” refers to a nucleic acid, amino acid, or non-peptide residue that can be inserted between one or more molecules. For example, linkers can be used to facilitate manipulation by providing desired regions of interest between components. Linkers can also be provided to enhance expression of the fusion protein from transformants and to reduce steric hindrance so that the component can assume its optimal tertiary structure and/or interact properly with the target molecule The linker sequence may include one or more amino acids naturally linked to the receptor component. It maybe also additional sequence to enhance expression of the fusion protein, or to provide a preferred site of interest specifically to enhance expression of the fusion protein, and/or added sequences used to enhance the interaction of the component with its target molecule.

Ideally, the mentioned linker can increase the flexibility of fusion proteins without interfering with the structure of each component of the fusion protein. In some embodiments, the linker residue is a peptide linker with 2 to 100 amino acid residues in length. Exemplary linkers include linear peptides with at least 2 amino acid residues, such as Gly-Gly (SEQ ID NO: 17), Gly-Ala-Gly (SEQ ID NO: 18), Gly-Pro-Ala (SEQ ID NO: 19), Gly-(G)n (SEQ ID NO: 20), and Gly-Ser (GS) (SEQ ID NO: 21) linkers. The GS linker disclosed herein includes (GS)n (SEQ ID NO: 21), (GSGSG)n (SEQ ID NO: 22), (G2S)n (SEQ ID NO: 23), G2S2G (SEQ ID NO: 24), (G2SG)n (SEQ ID NO: 25), (G3S)n (SEQ ID NO: 26), (G4S)n (SEQ ID NO: 8), (GGSGG)nGn (SEQ ID NO: 27), GSG4SG4SG (SEQ ID NO: 28), and (GGGGS)n (SEQ ID NO: 8), but is not limited thereto, where n is an integer of at least 1. An example of a (G)n linker is the G9 linker, and an example of a (GGGGS)n (SEQ ID NO: 5) linker includes GGGGS (SEQ ID NO: 8) or (GGGGS)3 (SEQ ID NO: 9) linker composed of suitable linear peptides including polyglycine, polyserine, polyproline, polyalanine, and oligopeptides composed of alanine and/or serine and/or proline and/or glycine amino acid residues. The linker residue can be used to connect the constituent components of the fusion protein disclosed herein.

In the present invention, the linker may be composed of an amino acid sequence with SEQ ID NO: 8 or SEQ ID NO: 9.

The fusion protein disclosed in this invention may or may not include a signal peptide that functions to secrete the fusion protein from the host cell, and the nucleic acid sequence encoding the signal peptide can be operably linked to the nucleic acid sequence encoding the protein of interest. In some embodiments, the fusion protein includes the signal peptide, while in others, it does not.

Furthermore, the fusion protein described in this invention can include modified forms of protein-binding peptides. For example, the fusion protein component can undergo post-translational modifications such as glycosylation, sialylation, acetylation, and phosphorylation for any arbitrary protein-binding peptide.

Unless otherwise stated, the fusion protein of this invention is administered as a polypeptide (or a nucleic acid encoding the polypeptide), which is not a part of a live, attenuated, or recombinant bacterial or viral vector vaccine. Additionally, unless otherwise specified, the fusion protein of this invention is a purified fusion protein and for example, it is not incorporated into the flagellum.

In the present invention, “fusion” refers to amalgamating two molecules with different or same functions or structures, by any physical, chemical, or biological means through which peptides can be connected. The fusion protein or polypeptide constituting the fusion protein can be produced by chemical peptide synthesis methods disclosed in the art, or by cloning the gene encoding the fusion protein by PCR (polymerase chain reaction) amplification or synthesis using the disclosed methods and expressing it in an expression vector.

In a particular embodiment of the present invention, the fusion protein may be composed of an amino acid sequence selected from a group consisting of SEQ ID NO: 10 to 16.

In the present invention, the term “metabolic syndrome” may also be referred to as metabolic disorders or diseases, and may be selected from a group consisting of diabetes, obesity, insulin resistance, fatty liver, hyperlipidemia, and hypertension. Preferably, the metabolic syndrome may be selected from a group consisting of diabetes, obesity, insulin resistance, and fatty liver.

In the present invention, the non-alcoholic fatty liver disease (NAFLD) can be selected from a group consisting of non-alcoholic steatosis, non-alcoholic steatohepatitis, fibrosis, and cirrhosis, and the most preferable one is non-alcoholic steatohepatitis.

The pharmaceutical composition of the present invention can be formulated in various dosage forms depending on the administration route, using a pharmaceutically acceptable carrier in accordance with a method disclosed in the art, in addition to the fusion protein. The term “pharmaceutically acceptable” refers to a non-toxic substance that is physiologically acceptable and does not interfere with the action of the active ingredient, and does not usually cause allergic reactions or similar reactions such as gastrointestinal disturbances or dizziness when administered to humans. Examples of such carriers include all types of solvents, dispersants, aqueous or oil-in-water emulsions, aqueous compositions, liposomes, microbeads, and microsomes.

The administration route can be oral or non-oral. Non-oral administration methods may include but are not limited to intravenous, intramuscular, intraarterial, intramedullary, intrathecal, intracardiac, transdermal, subcutaneous, intraperitoneal, intranasal, intragastric, local, rectal or intrauterine administration.

When the pharmaceutical composition of the present invention is administered orally, it can be formulated in various forms such as powders, granules, tablets, capsules, liquids, gels, syrups, suspensions, wafers, and the like according to methods known in the art using suitable oral delivery vehicles. Examples of suitable vehicles include sugars such as lactose, dextrose, sucrose, sorbitol, mannitol, xylitol, erythritol, and maltitol, as well as starches such as corn starch, wheat starch, rice starch, and potato starch, and cellulose derivatives such as cellulose, methylcellulose, sodium carboxymethylcellulose, and hydroxypropyl methylcellulose, and fillers such as gelatin and polyvinylpyrrolidone. In some cases, cross-linked polyvinylpyrrolidone, carrageenan, alginic acid, or sodium alginate may be added as a disintegrant. Additionally, the pharmaceutical composition may further include anti-adherents, lubricants, wetting agents, fragrances, emulsifiers, and preservatives as needed.

In addition, when administered non-orally, the pharmaceutical composition of the present invention can be formulated in the form of injectables, transdermal delivery agents, and nasal inhalants, along with suitable non-oral vehicles, according to methods known in the art. For injectables, the composition must be sterilized and protected from contamination by microorganisms such as bacteria and fungi. Suitable vehicles for injectables may include, but are not limited to, water, ethanol, polyols such as glycerol, propylene glycol, and liquid polyethylene glycol, mixtures thereof, and/or solvents or dispersing agents containing plant oils. Preferably, suitable vehicles include Hank's solution, Ringer's solution, PBS (phosphate-buffered saline) containing triethanolamine, or sterile injectable water, 10% ethanol, appearance solutions such as 40% propylene glycol and 5% dextrose. To protect the injectables from microbial contamination, various antimicrobial and antifungal agents such as parabens, chlorobutanol, phenol, sorbic acid, and thimerosal can be added. Additionally, most injectables can include isotonic agents such as dextrose or sodium chloride.

For the case of transdermal administration, the form of the pharmaceutical composition can include ointments, creams, lotions, gels, topical solutions, pastes, liniments, aerosols, and the like. The term “transdermal administration” as used herein refers to the delivery of the pharmaceutical composition to the skin locally, so that an effective amount of the active ingredient contained in the pharmaceutical composition is delivered into the skin. For example, the pharmaceutical composition of the present invention can be administered by preparing it in injectable form and injecting it lightly into the skin using a fine 30 gauge needle (prick), or by direct application to the skin. These forms are described in prescription references commonly known in pharmaceutical chemistry.

For the case of inhalation administration, the compound used according to the present invention can be conveniently delivered in aerosol spray form from a pressurized pack or a nebulizer with a suitable propellant, such as dichlorodifluoromethane, trichlorofluoromethane, dichlorotetrafluoroethane, carbon dioxide, or other suitable gas. The dosage units can be determined by providing a valve to deliver a metered amount. For example, gelatin capsules and cartridges for use in an inhaler or insufflator can be formulated to contain a powder mix of the compound and a suitable powder base, such as lactose or starch.

As other pharmaceutically acceptable carriers, reference may be made to those known in the art.

In addition, the pharmaceutical composition according to the present invention may further include one or more of a buffer (for example, saline or PBS), a carbohydrate (for example, glucose, mannose, sucrose, or dextran), an antioxidant, a bacteriostat, a chelating agent (for example, EDTA or glutathione), an adjuvant (for example, aluminum hydroxide), suspending agents, thickening agents and/or preservatives.

Furthermore, the pharmaceutical composition of the present invention can be formulated using methods known in the art to provide rapid, sustained or delayed release of the active ingredient after administration to a mammal.

Additionally, the pharmaceutical composition of the present invention can be co-administered with a disclosed substance having preventive or therapeutic effects on non-alcoholic fatty liver disease or metabolic syndrome.

The present invention provides a food composition for the prevention or improvement of non-alcoholic fatty liver disease or metabolic syndrome comprising a fusion protein comprising flagellin, a fragment or a variant thereof, and an immunoglobulin Fc region as an active ingredient.

The food composition of the present invention includes all forms of functional food, nutritional supplements, health food, and food additives. The food composition of the present invention can be manufactured in various forms according to conventional methods known in the art.

For example, as a health food, the fusion protein may be prepared in the form of tea, juice, and drinks containing the fusion protein for consumption, or in the form of granules, capsules, and powders for ingestion. Additionally, the food composition of the present invention can be manufactured in the form of a mixture containing the fusion protein and a substance or active ingredient known to have effects on non-alcoholic fatty liver disease or metabolic syndrome. For example, the food composition of the present invention may contain trace amounts of minerals, vitamins, lipids, sugars, and ingredients with preventative or therapeutic effects on non-alcoholic fatty liver disease or metabolic syndrome in addition to the fusion protein. The minerals may include calcium and iron, which are necessary nutrients for growth, while the vitamins may include vitamin C, vitamin E, vitamin B1, vitamin B2, and vitamin B6. The lipids may include alkoxyglycerol or lecithin, and sugars may contain fructooligosaccharides.

In addition, functional foods include beverages (including alcoholic beverages), fruits and their processed products (e.g., canned fruit, bottled fruit, jam, marmalade, etc.), fish, meat and their processed foods (e.g., ham, sausage corned beef). etc.), breads and noodles (e.g., udon, buckwheat noodles, ramen, spaghetti, macaroni, etc.), fruit juice, various drinks, cookies, taffy, dairy products (e.g. butter, cheese, etc.), edible vegetable oil, margarine, vegetable protein, Retort foods, frozen foods, various seasonings (e.g., soybean paste, soy sauce, sauce, etc.) can be prepared by adding the composition of the present invention.

The present invention provides a use of a fusion protein comprising flagellin, a fragment or a variant thereof, and an immunoglobulin Fc region for the preparation of an agent for the treatment of non-alcoholic fatty liver disease or metabolic syndrome.

The present invention also provides a method for treating non-alcoholic fatty liver disease or metabolic syndrome comprising administering an effective amount of a composition comprising a fusion protein comprising flagellin, a fragment or a variant thereof, and an immunoglobulin Fc region as an active ingredient to a subject in need thereof.

The term ‘effective amount’ in the present invention refers to an amount that exhibits an effect of improving, treating, detecting, diagnosing, or inhibiting or reducing the progression of non-alcoholic fatty liver disease or metabolic syndrome when administered to a subject, and the ‘subject’ refers to an animal, It may be preferably a mammal, especially an animal including a human, and may also be a cell, tissue, organ, etc. derived from an animal. The subject may be a patient in need of the effect.

The ‘treatment’ of the present invention refers comprehensively to improving symptoms due to non-alcoholic fatty liver disease or metabolic syndrome or non-alcoholic fatty liver disease or metabolic syndrome, which cures, substantially prevents, or improves the condition ameliorating, including but not limited to relieving, curing or preventing one or most of the symptoms resulting from the disease.

The term “comprising” used in this specification means the same as “including” or “characterized by”, and does not exclude additional components or method steps that are not specifically mentioned in the composition or method according to the present invention. Additionally, the term “consisting of” means that any additional elements, steps or components that are not specifically mentioned are excluded. The term “essentially consisting of” means that the composition or method may include additional components or steps that do not substantially affect the basic characteristics of the composition or method, in addition to the substances or steps that are specifically mentioned.

### Advantages of the Invention

The composition of the present invention has excellent effects in treating metabolic syndromes including obesity, insulin resistance, diabetes, non-alcoholic fatty liver disease, and can be very useful for developing prevention or treatment agents for non-alcoholic fatty liver disease or metabolic syndromes.

### BRIEF DESCRIPTION OF THE FIGURES

FIGS. 1A and 1B show the weight and dietary intake of the normal diet group (ND), high-fat diet group (HFD), and high-fat diet group with Fc-flagellin fusion protein administration (HFD+mIgG1-Fc2-Bsflagellin).

FIGS. 2A and 2B are diagrams showing the results of glucose tolerance tests (GTT) and insulin tolerance tests (ITT) of the normal diet group (ND), high-fat diet group (HFD), and high-fat diet group with Fc-flagellin fusion protein administration (HFD+mIgG1-Fc2-Bsflagellin).

FIG. 3 is a result of visual observation and weight measurement of the extracted liver of the general diet group (ND), high fat diet group (HFD), and high fat diet and Fc-flagellin fusion protein administration group (HFD+mIgG1-Fc2-Bsflagellin) after the experiment.

FIG. 4 is a diagram observed through a microscope after Oil-red O staining of the extracted liver tissue of the normal diet group (ND), high-fat diet group (HFD), and high-fat diet group with Fc-flagellin fusion protein administration (HFD+mIgG1-Fc2-Bsflagellin) at the end of the experiment.

### SPECIFIC DESCRIPTION FOR CARRYING OUT THE INVENTION

Hereinafter, the present invention will be described in detail through following examples. However, these examples are provided only to illustrate the present invention, and the present invention is not limited by them.

### Experimental Method:

### 1. Production of Fc-Flagellin Fusion Protein

In the present invention, a Fc-flagellin fusion protein was produced according to the method disclosed in Korean Patent Application No. 10-2020-0048368.

More specifically, a fusion protein (mIgG1-Fc2-Bsflagellin) was produced by fusing Bacillus subtilis flagellin (Bsflagellin) with linker (GGGGS) (SEQ ID NO: 8) and mIgG1 Fc (SEQ ID NO: 7) with SEQ ID NO: 11. This fusion protein was used in the present invention.

### 2. Establishment of a Non-Alcoholic Steatohepatitis (NASH) Animal Model

1) The mice were divided into three groups: normal diet (ND), high-fat diet with phosphate-buffered saline (HFD+PBS), and high-fat diet with mIgG1-Fc2-Bsflagellin (HFD+mIgG1-Fc2-Bsflagellin).
Fifteen C57BL/6J mice (4 weeks old, male) were purchased from Orient Bio.
A diet with 60% fat content (Orient Bio, catalog number: D12492) was purchased and used as a high-fat diet, and a diet with 10% fat content (Orient Bio, catalog number: 5L79) was purchased and used as a normal diet.

2) The mice used in the experiment were divided into 5 mouse per group with the body weight as similar as possible.

3) mIgG1-Fc2-Bsflagellin fusion protein (5 ug/16 ul/mouse) was administered intranasally to mice 2 weeks before feeding the high-fat diet and then administered a total of 12 times.
To the mice of the normal diet group (ND) and the high fat diet group (HFD), 16 ul of phosphate-buffered saline (PBS) was administered intranasally.

4) The high-fat diet and the normal diet were supplied fresh every week at 40 g per mouse.
A high-fat diet was supplied to the mice for a total of 10 weeks starting 2 weeks after administration of protein and saline into the mice's nasal cavity.

5) The mice's body weights and food intake were observed and observed every week.
The high-fat diet was stopped when there was a significant difference in body weight between the high-fat diet group and the normal diet group.

### 3. Glucose-Tolerance Test (GTT)

1) Prior to performing the GTT, the mice were fasted for approximately 16 hours.
During the fasting period, the mice were housed in cages without bedding and provided with only water.

2) The next day, the mice were weighed.

3) Before injecting glucose (D-(+)-Glucose, Sigma, catalog number: G7021) into the mice, fasting blood glucose levels were measured by collecting blood from the tail vein.
Blood glucose levels were measured using a glucometer (Gluneo Lite, OSANG Healthcare).

4) After measuring fasting blood glucose, the amount of 20% glucose solution to be injected was calculated based on the weight of the mouse and injected into the abdominal cavity of the mouse.
The amount of glucose to be injected into mice was intraperitoneally injected with 2 g of 20% D-glucose solution per kg of mouse body weight.

5) Blood glucose levels were measured by collecting blood from the tail vein at 15, 30, 60, 90, and 120 minutes after glucose injection.

### 4. Insulin Tolerance Test (ITT)

1) Prior to performing the ITT experiment, mice were fasted for approximately 4 hours.
During fasting, mice were kept in a cage without bedding and provided only water.

2) After 4 hours, the mice were weighed.

3) Prior to injecting insulin (Sigma, catalog number: 19278) into the mouse, fasting blood glucose levels were measured by collecting blood from tail vein of the mouse.

4) After measuring fasting blood sugar, the amount of insulin to be injected was calculated based on the weight of the mouse and injected into the abdominal cavity of the mouse.
The dosage of insulin injected into the mouse was 0.75 IU (international units) per kg of mouse.

5) Blood glucose levels were measured by collecting blood from the mouse tail vein at 15, 30, 60, 90, and 120 minutes after insulin injection.

### 5. Measurement of Organ Weight

After the experiment was terminated, the mice were sacrificed, and several organs were extracted and the weight of each organ was measured.

### 6. H&E Staining of Tissue Samples

\textcircled{1} The extracted organs were fixed in neutral-buffered formalin.

\textcircled{2} Organs that had been fixed were trimmed to a thickness of about 2 to 3 mm in a size suitable for tissue specimen production.

\textcircled{3} The specimens, which were trimmed appropriately for tissue specimen production, were placed in a cassette with an individual number and subjected to tissue processing (STP120 Spin tissue Processor, Myr) for 13 hours.

\textcircled{4} Sections generated by cutting to a thickness of about 3 μm using a microtome (Finesse ME Microtome, Thermo Shandon) were attached to a slide, dried, deparaffinized, and washed with distilled water after dehydration.

\textcircled{5} Hematoxylin and eosin staining was performed.

### 7. Oil Red O Staining Sample Preparation

\textcircled{1} The extracted organs were fixed in neutral buffered formalin.

\textcircled{2} The fixed organs were sectioned to a thickness of about 2 to 3 mm in a size suitable for tissue specimen production.

\textcircled{3} Specimens trimmed appropriately for tissue specimen production were treated with 30% Sucrose (Merck) solution. After the sucrose treatment was completed, a cryoblock was made using OCT Compound (Tissue Tek O.C.T Compound), and then sections were prepared using a cryostat (CM3000, Leica), attached to slides, and dried.

\textcircled{4} Oil red O staining was performed.

### Experimental Results

### 1. Effect of Preventing Obesity by a High-Fat Diet (HFD)

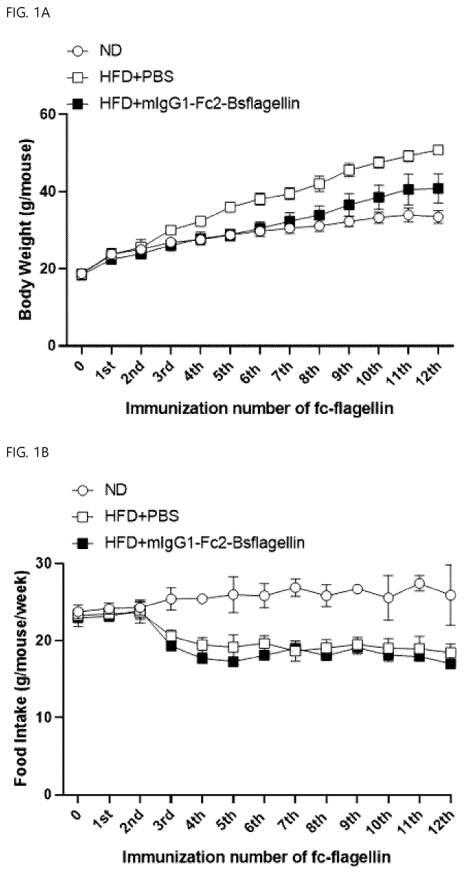

It was confirmed that the increase in body weight was significantly increased in mice fed HFD compared to mice fed normal diet (NA). When mIgG1-Fc2-Bsflagellin was intranasally administered every week to a group of mice fed a high-fat diet, it was confirmed that fat-induced obesity was suppressed. Food intake decreased in the mouse group fed the high-fat diet compared to the normal diet, but there was no effect on the degree of obesity (FIGS. 1A and 1B).

### 2. Improvement in Glucose Tolerance Test (GTT) and Insulin Tolerance Test (ITT)

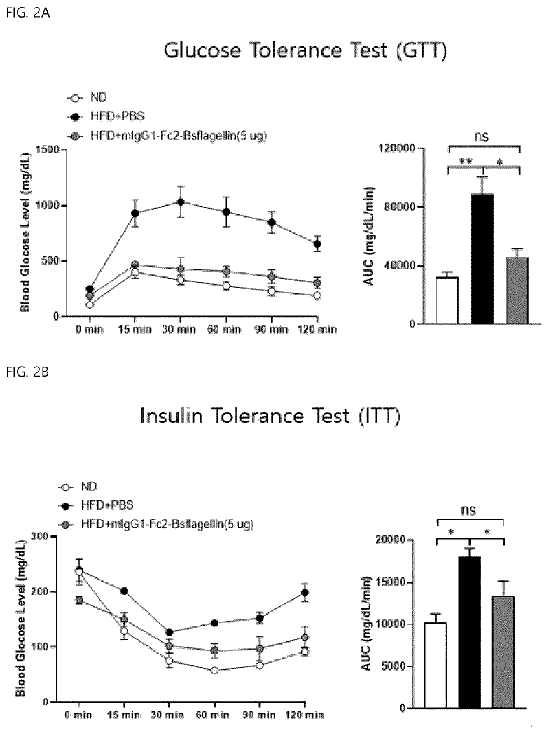

Higher tolerance was observed in GTT and ITT in the high-fat diet group than in the normal diet group, but in the group administered with mIgG1-Fc2-Bsflagellin, both GTT and ITT were confirmed to be improved to the level of the normal diet group (FIGS. 2A and 2B). This suggests that metabolism can be controlled at a normal value very similar to that of the general diet group, and suggests that there is potential as a treatment for metabolic diseases such as diabetes.

### 3. Improvement in Hepatic Steatosis

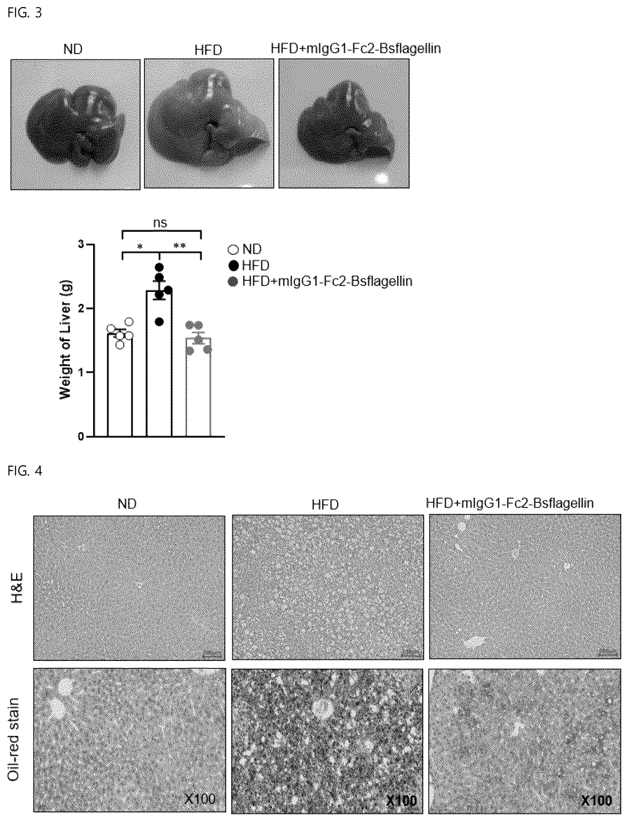

It was confirmed that the weight of the liver increased in the high-fat diet group was improved similarly to normal by administration of mIgG1-Fc2-Bsflagellin (FIG. 3).

Additionally, it was confirmed that hepatic steatosis (red color) induced by the HFD was improved by Fc-flagellin administration (FIG. 4).

The amino acid sequences of each protein (or peptide) in the present invention are as follows:
Bacillus subtilis flagellin (BSflagellin) amino acid sequence: SEQ ID NO: 1
Salmonella dublin flagellin (Sdflagellin) amino acid sequence: SEQ ID NO: 2
Pseudomonas aeruginosa flagellin (Paflagellin) amino acid sequence: SEQ ID NO: 3
Shigella flexneri flagellin (Shflagellin) amino acid sequence: SEQ ID NO: 4
Escherichia coli flagellin (Ecflagellin) amino acid sequence: SEQ ID NO: 5
Human IgG4 Fc amino acid sequence: SEQ ID NO: 6
Mouse IgG1 Fc amino acid sequence: SEQ ID NO: 7
Linker 1 amino acid sequence: SEQ ID NO: 8
Linker 2 amino acid sequence: SEQ ID NO: 9
hIgG4-Fc-Bsflagellin fusion protein amino acid sequence: SEQ ID NO: 10
mIgG1-Fc-Bsflagellin fusion protein (Linker 1) amino acid sequence: SEQ ID NO: 11
mIgG1-Fc-Bsflagellin fusion protein (Linker 2) amino acid sequence: SEQ ID NO: 12
hIgG4-Fc-Sdflagellin fusion protein amino acid sequence: SEQ ID NO: 13
hIgG4-Fc-Paflagellin fusion protein amino acid sequence: SEQ ID NO: 14
hIgG4-Fc-Shflagellin fusion protein amino acid sequence: SEQ ID NO: 15
hIgG4-Fc-Ecflagellin fusion protein amino acid sequence: SEQ ID NO: 16

### INDUSTRIAL APPLICABILITY

The composition of the present invention is very effective in treating obesity, insulin resistance, diabetes, metabolic syndrome including fatty liver and non-alcoholic fatty liver disease. Industrial applicability is very high as it can be used very usefully in the development of prevention or treatment of non-alcoholic fatty liver disease or metabolic syndrome.

1. A pharmaceutical composition for the prevention or treatment of non-alcoholic fatty liver disease or metabolic syndrome comprising as an active ingredient a fusion protein comprising flagellin, a fragment or a variant thereof and an immunoglobulin Fc region.

2. The pharmaceutical composition according to claim 1, wherein the flagellin is a flagellin derived from a microorganism selected from the group consisting of Bacillus, Salmonella, Helicobacter, Vibrio, Serratia, Shigella, Treponema, Legionella, Borrelia, Clostridium, Agrobacterium, Bartonella, Proteus, Pseudomonas, Escherichia, Listeria, Yersinia, Campylobacter, Roseburia, and Marinobacter.

3. The pharmaceutical composition according to claim 1, wherein the flagellin is a flagellin derived from a selected group of microorganisms including Salmonella enteritidis, Salmonella typhimurium, Salmonella dublin, Salmonella enterica, Helicobacter pylori, Vibrio cholera, Vibrio vulnificus, Vibrio fibrisolvens, Serratia marcesens, Shigella flexneri, Treponema pallidum, Borrelia burgdorferi, Clostridium difficile, Agrobacterium tumefaciens, Bartonella clarridgeiae, Proteus mirabilis, Bacillus subtilis, Bacillus cereus, Bacillus halodurans, Pseudomonas aeruginosa, Escherichia coli, Listeria monocytogenes, Yersinia pestis, Campylobacter spp, Roseburia spp, and Marinobacter spp.

4. The pharmaceutical composition according to claim 1, wherein the flagellin comprises a conserved sequence recognized by toll-like receptor 5 (TLR5).

5. The pharmaceutical composition according to claim 1, wherein the fragment has a hypervariable region removed from wild-type flagellin.

6. The pharmaceutical composition according to claim 1, wherein the fragment comprises at least one domain selected from the group consisting of C-terminal domain 0, C-terminal domain 1, C-terminal domain 2, N-terminal domain 2, N-terminal domain 1, N-terminal domain 0 of wild type flagellin, and a domain having 80% or greater amino acid sequence homology with the said domain.

7. The pharmaceutical composition according to claim 1, wherein the variant exhibits 80% or more amino acid sequence homology with wild-type flagellin and exhibits Toll-like receptor 5 (TLR5) stimulating activity.

8. The pharmaceutical composition according to claim 1, wherein the immunoglobulin Fc region is derived from the Fc of human or animal immunoglobulin IgG, IgM, IgD, IgA or IgE.

9. The pharmaceutical composition according to claim 1, wherein the immunoglobulin Fc region is derived from the Fc of human or animal immunoglobulin IgG1, IgG2, IgG3 or IgG4.

10. The pharmaceutical composition according to claim 1, wherein the immunoglobulin Fc region comprises at least one selected from the group consisting of CH1, CH2, CH3, and CH4 domains.

11. The pharmaceutical composition according to claim 1, wherein the N-terminus or C-terminus of the flagellin, a fragment thereof or a variant thereof is linked to the N-terminus or C-terminus of the immunoglobulin Fc region.

12. The pharmaceutical composition according to claim 1, wherein the flagellin, fragment thereof or variant thereof, and the immunoglobulin Fc region are connected by a linker.

13. The pharmaceutical composition according to claim 1, wherein the flagellin, the fragment or the variant thereof consists of an amino acid sequence selected from the group consisting of SEQ ID NO 1 to 5 or an amino acid sequence showing 80% or more sequence homology thereto.

14. The pharmaceutical composition according to claim 1, wherein the immunoglobulin Fc region consists of an amino acid sequence of SEQ ID NO: 6 or SEQ ID NO: 7.

15. The pharmaceutical composition according to claim 12, wherein the linker consists of an amino acid sequence of (GGGGS)n (n is 1 to 5).

16. The pharmaceutical composition according to claim 1, wherein the fusion protein consists of an amino acid sequence selected from the group consisting of SEQ ID NO: 10 to 16.

17. The pharmaceutical composition according to claim 1, wherein the metabolic syndrome is selected from the group consisting of diabetes, obesity, insulin resistance, fatty liver, hyperlipidemia and hypertension.

18. The pharmaceutical composition according to claim 1, wherein the non-alcoholic fatty liver disease is non-alcoholic steatosis, non-alcoholic steatohepatitis, fibrosis or cirrhosis is characterized in that it is non-alcoholic steatosis, non-alcoholic steatohepatitis, fibrosis or cirrhosis.

19. A food composition for the prevention or improvement of non-alcoholic fatty liver disease or metabolic syndrome comprising a fusion protein comprising flagellin, a fragment or a variant thereof, and an immunoglobulin Fc region as an active ingredient.

20. (canceled)

21. A method for treating non-alcoholic fatty liver disease or metabolic syndrome, the method comprising administering an effective amount of a composition comprising a fusion protein comprising flagellin, a fragment or a variant thereof, and an immunoglobulin Fc region as an active ingredient to a subject in need thereof.
